(19) United States
(12) Patent Application Publication
(10) Pub. No.: US 2010/0284854 A1
(43) Pub. Date: Nov. 11, 2010

(54) Radicals on eradication surfaces

(76) Inventor: John Lee Edwards, Sarasota, FL (US)

Correspondence Address: Jackson Walker L.L.P.;c/o Mark A. Tidwell, Suite 1900, 1401 McKinney Street, Houston, TX, 77010, US

(21) Appl. No.: 12/006,676

(22) Filed: Dec. 31, 2007

Related U.S. Application Data
(60) Provisional application No. 60/878,184, filed on Jan. 3, 2007.

(51) Int. Cl. A61L 9/20; B01J 19/08

(52) U.S. Cl. 422/4; 422/186.3

(57) ABSTRACT

Disclosed are a method and apparatus with several embodiments that use low output UV energy to create hydroxyl radicals on photocatalytic semiconductor coated surfaces in order to produce hydroxyl radicals to structurally eradicate contaminants impacting those surfaces. Low wattage UVA black-light white and black-light blue plasma lamps are used to activate photocatalytic semiconductor coatings on solid and mesh surfaces constructed of fluropolymers (Teflon®), of aluminum, of stainless steel, and coatings on the outer lamp wall of the UVA lamp itself. The embodiments show that sufficient UVA energy is being transmitted through solid lamp wall, through the photocatalytic semiconductor coating on the outer lamp wall (from beneath), and through multiple layers of coated fluropolymer, aluminum, and stainless steel mesh to create hydroxyl radicals on the surfaces of downstream layers. The current carrying foundation materials, to include electro-conductive polymer, can be voltage charged to attract pathogens with natural cell surface charges to the coated eradication surfaces. The method and apparatus may be used to create radicals on eradication surfaces to structurally dismantle and destroy contaminant particles, including lethal virus, bacteria, mycotoxin, spores, odor molecules, allergens, smoke particles, and industrial pollutants.

### CROSS REFERENCE TO RELATED APPLICATIONS

This application claims priority from provisional application filing No. 60/878,184 “ViraClean 1” filed Jan. 3, 2007, Utility application filing Ser. No. 11/642,292 “Consumer Virus Eradicator” filed Dec. 20, 2006, provisional application filing No. 60/751,546 “Consumer Virus Eradicator” filed Dec. 20, 2005 and provisional application filing No. 60/751,547 “Airborne Virus Eradicator” filed Dec. 20, 2005.

### BACKGROUND OF THE INVENTION

This invention relates broadly to air cleaning devices that remove airborne contaminants of many types, including, viruses, bacteria, mycotoxin, fungi, spores, allergen particles, odiferous molecules, Volatile Organic Compounds, and other Toxic Industrial Chemicals. More specifically, the invention relates to a device that utilizes low power, UVA wavelength electromagnetic energy to generate hydroxyl radicals on the surface of photocatalytic semiconductor coated eradication surfaces, thereby facilitating the removal of airborne contaminants with technology that is safer for consumer and health care applications, and consumes less energy.

There is a critical need to improve the air quality of inhabited areas by eliminating suspended contaminants. Sale of consumer air purifiers have risen dramatically worldwide since Sep. 11, 2001, because of civilian concern for epidemic pathogens (Avian Virus, SARS, Influenza), and their bio-engineered military grade counterparts (Plague, Smallpox). Currently popular electrostatic precipitation devices, supplemented by low level ozone or germicidal radiation, are only moderately effective for eradication pathogens.

Hydroxyl radicals are very potent one-electron oxidants that steal hydrogen molecules from passing organic material, as they seek to replace their missing covalent electron, leaving decayed carbon ions in the organic matter, destroying the pathogen.

Several compositions of nanocrystal based semiconductor formulations, including single and multiple layer sol gel based foundations, are known in the art to express hydroxyl radicals on and within interstitial cavities of three dimensional, fixed porous matrix coatings via the dissociation of H₂O water ambient humidity molecules into powerful, short lived .OH hydroxyl radicals. The water vapor is consumed, but rapidly cycled back to natural elements. The end result of this photochemical conversion of pathogen material for air purification is eventually water and virtually undetectable amounts of decayed carbon based material, so that the purification is also a bio-friendly process.

The presence of hydroxyl radicals on nanocrystal semiconductor surfaces can be confirmed by the use of a clear, colorless chromophore, Nitroblue Tetrazolium (“NBT”). NBT is a chemical substrate used in medical research to measure the bacterial killing ability of human neutrophils, primarily whether they are producing phagocytic singlet oxygen ₁O², normally generated in such healthy white blood cells of the immune system. Singlet oxygen is a powerful, endogenous oxidant, second to hydroxyl radicals in its thermodynamic oxidation potential. Singlet oxygen and hydroxyl radicals will both reduce colorless NBT solution to an indigo dye-based chloride salt precipitate, thereafter displaying a deep blue visible color if either has been present to cause the reaction.

### BRIEF SUMMARY OF THE INVENTION

The present invention shows the creation of hydroxyl radicals using photocatalytic semiconductor coatings on surfaces that can be used to molecularly dismantle and eradicate pathogens and other contaminants suspended in an airstream impacting the coated surface. The invention uses electromagnetic energy from comparatively low output “black light white” UVA lamps in the ultraviolet 315 nm to 380 nm wavelength range to generate hydroxyl radicals on the photocatalytic semiconductor coated surfaces. Also disclosed is the activation of multiple layers of coated surfaces after the UVA energy has already passed through other coated solid and multiple mesh forms of materials that are semi-transparent to the activating energy wavelengths, but whose eradication coatings are activated by the energy passing through them. Such intermediate coated surface layers do not absorb all of the passing energy for activation of their own coating, but pass on the energy balance to subsequent layered surfaces. This permits the creation of more effective, multilayered purification devices.

Accordingly, it is a primary object of the present invention to provide a new and improved method and apparatus for creating free radical rich surfaces to destroy contaminants thereby, using low power UVA energy.

It is a further object of this invention to improve breathable air by using such activated surfaces and in so doing, to use no ingredients and leave no residue that could be harmful to the environment.

It is a further object of this invention to use the activated surfaces of this invention to eradicate impacting contaminants by structural dissociation of their cellular and chemical makeup, converting them to non-threatening forms, down to the level of natural environmental molecules.

It is a further object of this invention to provide several apparatus embodiments of the activated eradication surfaces for carrying out methods that achieve the foregoing objects and which are relatively simple in construction and effective in operation.

These and other objects will be made manifest when considering the following detailed specification when taken in conjunction with the appended drawing figures.

### BRIEF DESCRIPTION OF THE DRAWINGS

FIG. 1 is a chart showing the UV energy output of a 50 watt UVA black-light white lamp, also comparing it to the UV energy through an uncoated sheet of 0.9 millimeter thick fluropolymer (Teflon®).

FIG. 2 is the drawing of an image confirming free radical production on a length of photocatalytic semiconductor coated fluropolymer (Teflon®) tubing by a pattern of clear painted Nitroblue Tetrazolium that displays color after the lamp illuminated the coated surface.

FIG. 3 is a drawing of one embodiment of the invention showing a photocatalytic semiconductor lined cylinder housed in an air purifier chassis.

FIG. 4 is a chart showing the UV energy output of a UVA black-light white lamp of the FIG. 3 embodiment before and after permanent bonding of the proprietary photocatalytic semiconductor coating to the lamp's outer wall.

FIG. 5 is a drawing confirming free radical production on the photocatalytic coated outer lamp wall itself by a pattern of clear painted Nitroblue Tetrazolium that displayed color after the lamp was turned on, having illuminated the coated outer layer from beneath, through the glass lamp wall, and through the three dimensional sol gel based photocatalytic semiconductor coating.

FIG. 6 is a drawing image confirming free radical production on the positively charged photocatalytic semiconductor coated aluminum inner liner of the embodiment of FIG. 3 by a pattern of clear painted Nitroblue Tetrazolium that displayed color after the lamp was turned on.

FIG. 7 is a drawing showing a second embodiment of the invention with a folded, fine mesh constructed of photocatalytic semiconductor coated fluropolymer (Teflon®), inserted at the outlet of a filter cartridge in an air purifier chassis, such cartridge containing a black-light blue UVA lamp within, with the word BACK appearing on the surface of the coated mesh outlet when the clear painted Nitroblue Tetrazolium was activated by hydroxyl radicals produced thereon, and displayed the color pattern shown.

FIG. 8 is the drawing of an image showing the mesh of FIG. 7 unfolded to show that both the word FRONT, which was clear painted on the side positioned next to the black-light blue UVA lamp within the filter cartridge of FIG. 7, and the clear painted word BACK, which was positioned after the Front layer, showing when the mesh was unfolded that both layers were activated by free radicals produced thereon from UVA energy passing through both adjacent layers.

FIG. 9 shows the spectral emission of the UVA black-light blue lamps of the embodiment of FIG. 7.

FIG. 10 is the drawing of another embodiment of the invention showing a dismantling cartridge loaded into an air purifier chassis with cover removed, such cartridge containing two photocatalytic semiconductor coated black-light white UVA lamps positioned within the cartridge between two outer, photocatalytic semiconductor coated dielectric fiber dismantling mesh screens located at the cartridge entrance and exit.

FIG. 11 is the drawing of an image confirming production of free radicals on the rear exit dismantling mesh of the FIG. 11 cartridge, with the cross marks appearing when the clear painted Nitroblue Tetrazoliumon on the dielectric mesh was activated by hydroxyl radicals produced thereon and displayed the color pattern shown.

FIG. 12 is a drawing showing free radical creation confirmed by clear Nitroblue Tetrazolium pattern on two each layers of two separate extremely low porosity grades of photocatalytic coated fluropolymer (Teflon®) mesh and aluminum mesh where the mesh layers displaying “1” were positioned between the black-light white photocatalytic semiconductor coated UV lamp and mesh layers displaying “2”.

FIG. 13 shows the transmission of UV energy by the coated 50 watt UVA lamp through one layer of uncoated fluropolymer mesh, and separately shows the UV energy transmission through two coated layers of the fluropolymer mesh of FIG. 8.

FIG. 14 shows the transmission of UV energy by a coated 50 watt UVA lamp through two each layers of photocatalytic semiconductor bonded over fluropolymer coated stainless steel mesh, without any voltage charge applied to the metal core of either layer.

### DETAILED DESCRIPTION OF THE INVENTION

This application claims priority from provisional application filing No. 60/878,184 “ViraClean 1” filed Jan. 3, 2007, Utility application filing Ser. No. 11/642,292 “Consumer Virus Eradicator” filed Dec. 20, 2006, provisional application filing No. 60/751,546 “Consumer Virus Eradicator” filed Dec. 20, 2005 and provisional application filing No. 60/751,547 “Airborne Virus Eradicator” filed Dec. 20, 2005.

When activated by ultraviolet radiation with wavelengths shorter than 387.5 nm certain semiconductor nanoparticles produce a powerful molecular dismantling mechanism, hydroxyl radicals from ambient water molecules The hydroxyl radicals rob target molecule electrons from hydrogen atoms in organic matter they contact, in order to restore their own covalent electrons lost to the energizing radiation. A colloidal suspension coating of photocatalytic semiconductor nanoparticles known in the art, suspended in a sol gel based fixed matrix, provides a stable, yet porous dry carrier structure to permanently suspend the nanoparticles, while permitting target molecules to move freely in and out of the nanoparticle matrix. Hydroxyl radicals produced in the matrix will dismantle and eradicate pathogens impacting them.

This invention uses photocatalytic surfaces, including solid apparatus liners, lamp surfaces, and UV transmissive meshes, coated with an outer layer of photocatalytic semiconductor nanoparticles, to generate hydroxyl radicals on the surfaces thereof. These dismantling surfaces may be illuminated from within the apparatus by UV illumination from lamps radiating UVA (315-380 nm), UVB (280-315 nm), and UVC (200-280 nm) wavelengths to activate the photocatalytic coatings, since all these lamps radiate photon wavelengths shorter than 387.5 nm.

However, our research also shows that ultraviolet radiation from comparatively low wattage UVA lamps alone with most of their energy transmitted in the 315-380 nm range is very satisfactory for generating the photocatalytic eradication mechanism of this invention.

The UV transparent dismantling meshes, or coatings on other substrate material, are ideally composed of extruded or expanded tetrafluoroethylene, (trade name Teflon®), to serve as a compatible support structure for the photocatalytic semiconductor dismantling coating. The material's transmissivity to the activating UV bandwidth, its extreme inertness to chemicals, its low coefficient of friction, its ability to resist adhesion to almost any material, and its ability to withstand extremely high operating temperatures (thousands of degrees Celsius), render it generally ideal for applications involving treatment of corrosive, dismantling, or combustible air flows.

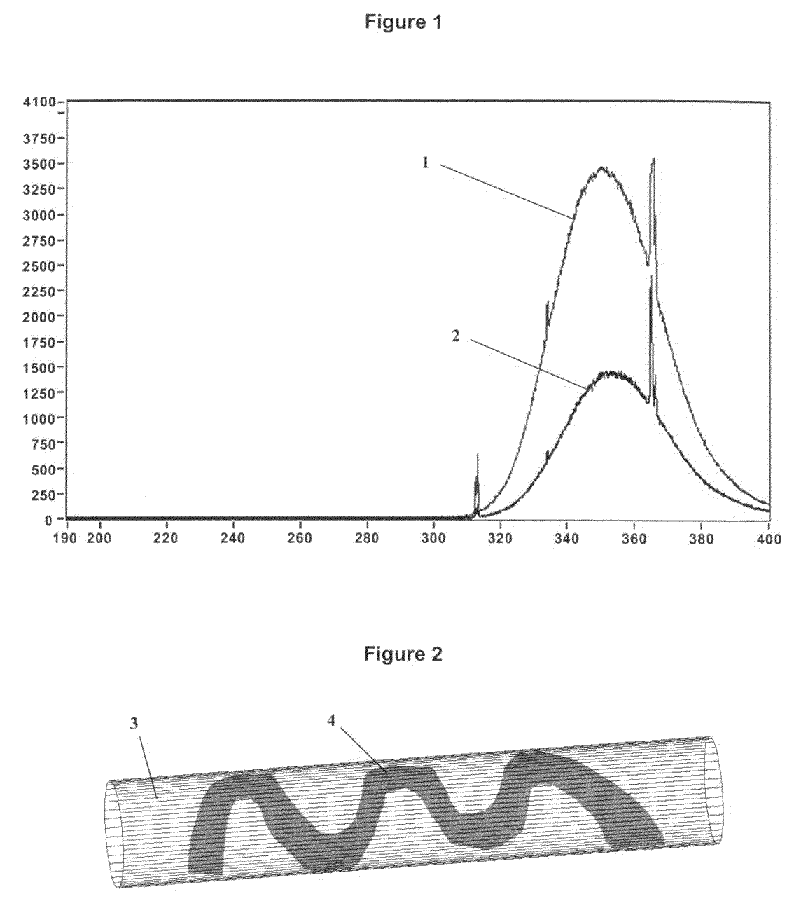

FIG. 1 is a chart showing the UV energy output 1 of a 50 watt UVA black-light white lamp, (FUL50TBL), comparing it to the UV energy 2 after passing through an uncoated sheet of 0.9 millimeter thick tetrafluoroethylene (Teflon®). The solid fluropolymer transmits approximately 42% of the incident UVA radiation.

Tetrafluoroethylene tubing is also a suitable substrate itself for photocatalytic semiconductor bonding to produce free radicals as shown by this inventor's previous disclosures. FIG. 2 is the drawing of an image confirming free radical production on a length of photocatalytic semiconductor coated fluropolymer tubing 3 by a pattern of clear painted Nitroblue Tetrazolium that displays color 4 after the lamp has illuminated the coated surface from the outside. Comparable results are obtained by illuminating the fluropolymer tube from within.

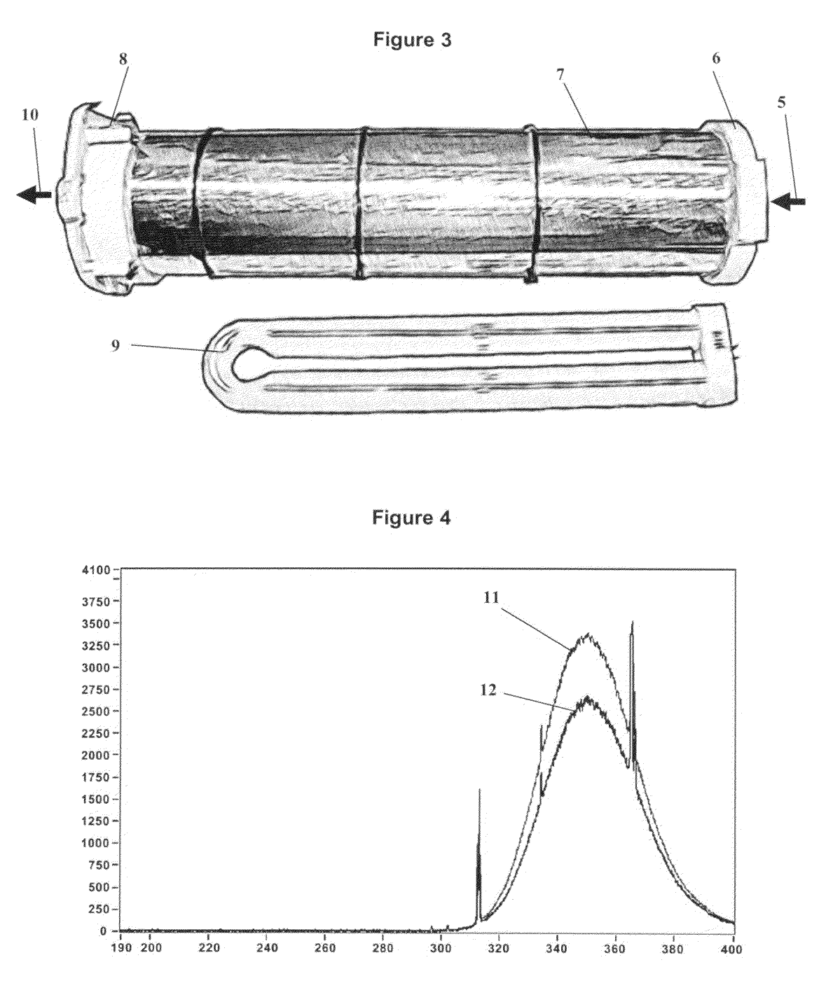

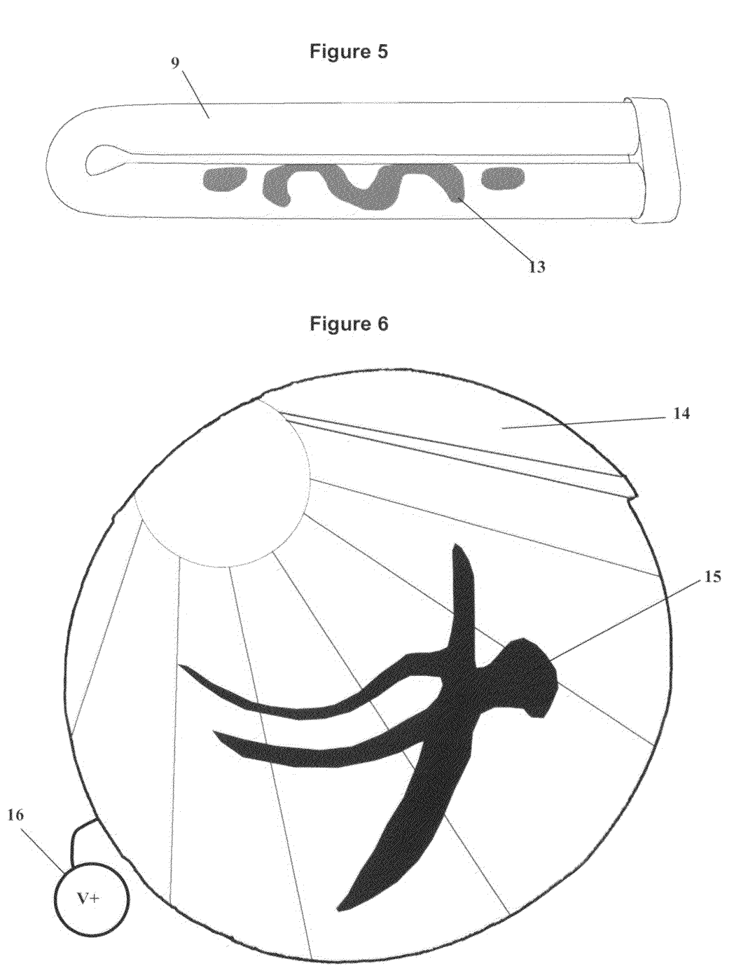

FIG. 3 is the drawing of an image showing a cylinder embodiment of this invention. Contaminated air 5 is drawn into the bottom inlet 6 of the reaction cylinder 7 by a fan (not shown) at the top 8 of the cylinder. Carried by the moving air, the suspended contaminants pass over, and many will impact, the photocatalytic coated surface of the wall of the illuminating lamp 9 which is inserted in cylinder 7. Purified air 10 exits the apparatus. FIG. 4 is a chart showing the UV energy output of a typical 40 watt UVA black-light white lamp, (B4040/0410 Seo Kang, Stinger), before 11 and after 12 the proprietary photocatalytic semiconductor coating is bonded to the lamp's outer wall. In our research the photocatalytic semiconductor coating absorbs 20% or less of the UV energy and passes on the rest. FIG. 5 is a drawing confirming that free radicals are being produced on the photocatalytic semiconductor coated outer lamp wall 9 of FIG. 3 by a pattern of clear painted Nitroblue Tetrazolium that displayed color 13 after the lamp was turned on. Note that the lamp wall outer coating is being activated by energy from beneath, from within the plasma lamp, UVA electromagnetic energy traveling through the glass lamp wall, and through the three dimensional sol gel based photocatalytic semiconductor coating as well.

Surviving contaminants that impact the photocatalytic coated aluminum inner cylinder liner are also eradicated. FIG. 6 is a drawing image confirming free radical production on the photocatalytic semiconductor coating 14 on aluminum inner liner of cylinder 7 of the embodiment of FIG. 3 by a pattern of clear painted Nitroblue Tetrazolium that displayed color 15 after the lamp (removed in this image) was turned on.

Pathogens are attracted to the current carrying liner of FIG. 6 by a positive or negative voltage potential imposed by a voltage-generator 16. Most suspended contaminants have a natural electrostatic charge present on them. Large macromolecules generally have a natural positive charge. For example, many allergens and dust particles tend to be positively charged, which helps keep these large particles suspended in air longer. Small microorganism particles typically have a negative charge on them. Bacteria and their fragments are negatively charged due to their cell wall chemistry. Gram negative bacteria contain negatively charged —COO* groups associated with proteins and lipopolysaccharides in their cell walls. Gram-negative bacterial endotoxins (pyrogens) are also negatively charged as are viruses and most colloids.

The voltage imposed liner 14 is coated with an inorganic dielectric such as tetrafluoroethylene, beneath the photocatalytic over coating. The photocatalytic semiconductor coating of FIG. 6 must be dielectrically insulated from its metal under layer, otherwise, the surface field of dismantling free radicals on the photocatalytic surface would self-quench via electrical charging of the embedded nanocrystals toward either polarity. The dielectric insulator layer must be inorganic because free radicals on the coated surface would attract and degrade an organic dielectric, just as they degrade and destroy organic pathogens. The process of permanently coating tetrafluoroethylene to the underlying current carrying metal structure, and the process of permanently bonding the photocatalytic colloidal matrix coating to the insulating dielectric layer, are proprietary and not disclosed here. In operation, the field of free radicals on the photocatalytic coated surfaces structurally dismantle the impacting contaminants, which are being held at their impact site by the attraction of the electric field under layer interacting with the electrostatic surface charge of opposite polarity naturally occurring on the cell surface of attracted pathogens.

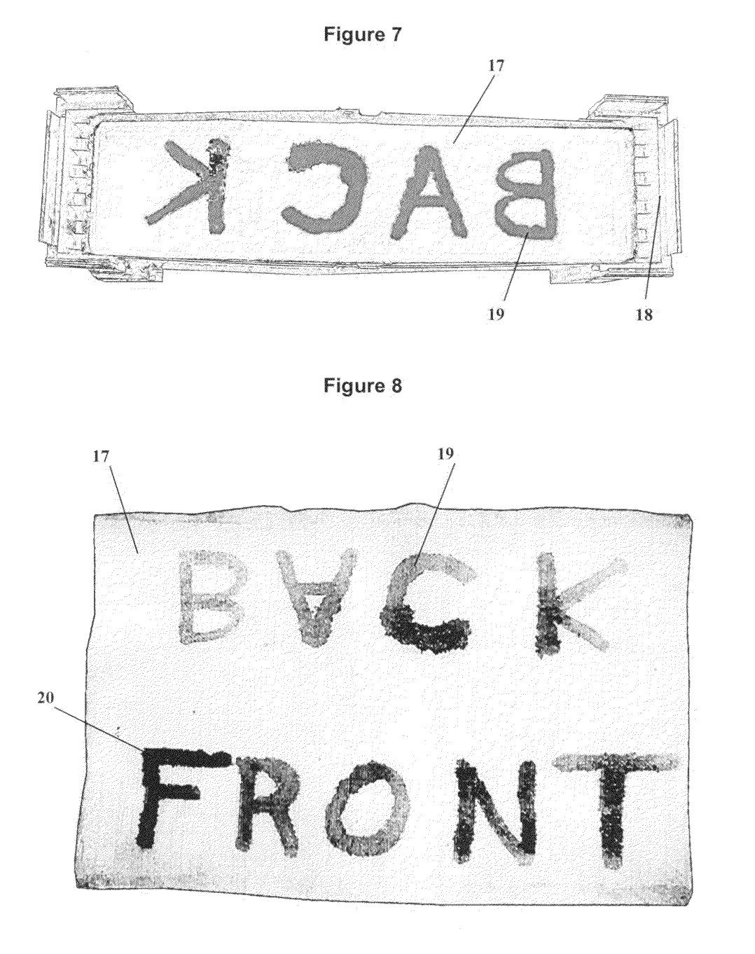

FIG. 7 is a drawing showing a second embodiment of the invention with a folded mesh 17 constructed of photocatalytic semiconductor coated fluropolymer (Teflon®), inserted at the outlet of a filter cartridge 18 in an air purifier chassis (not shown), such cartridge containing two uncoated 11 watt black-light blue UVA lamps within, (FPX11BLB, not shown) with the word BACK 19 appearing on the surface of the coated mesh outlet when the clear painted Nitroblue Tetrazolium was activated by hydroxyl radicals produced thereon, and displayed the color pattern shown.

FIG. 7 shows that the lamps create hydroxyl radicals on photocatalytic extruded fluropolymer (Teflon®) mesh 17, directly on a mesh adjacent to the lamp, and through that first mesh to a second mesh, where the first folded over half of the mesh is positioned between the UVA lamp and the second half mesh fold. The mesh used in this embodiment is constructed of solid PTFE extruded monofilament (FEP) of 279 microns diameter, not a coated PTFE fabric. The mesh opening of this embodiment is 450 microns for an open area of 39% before photocatalytic semiconductor solgel coating.

FIG. 8 is the drawing of an image showing the mesh of FIG. 7 unfolded to show that both the word FRONT 20, which was clear painted on the side positioned next to the black-light blue UVA lamp within the filter cartridge of FIG. 7, and the clear painted word BACK 19, which was positioned after the Front layer when folded over. The unfolded mesh confirms that both layers were activated by free radicals produced thereon from UV energy passing through both adjacent layers.

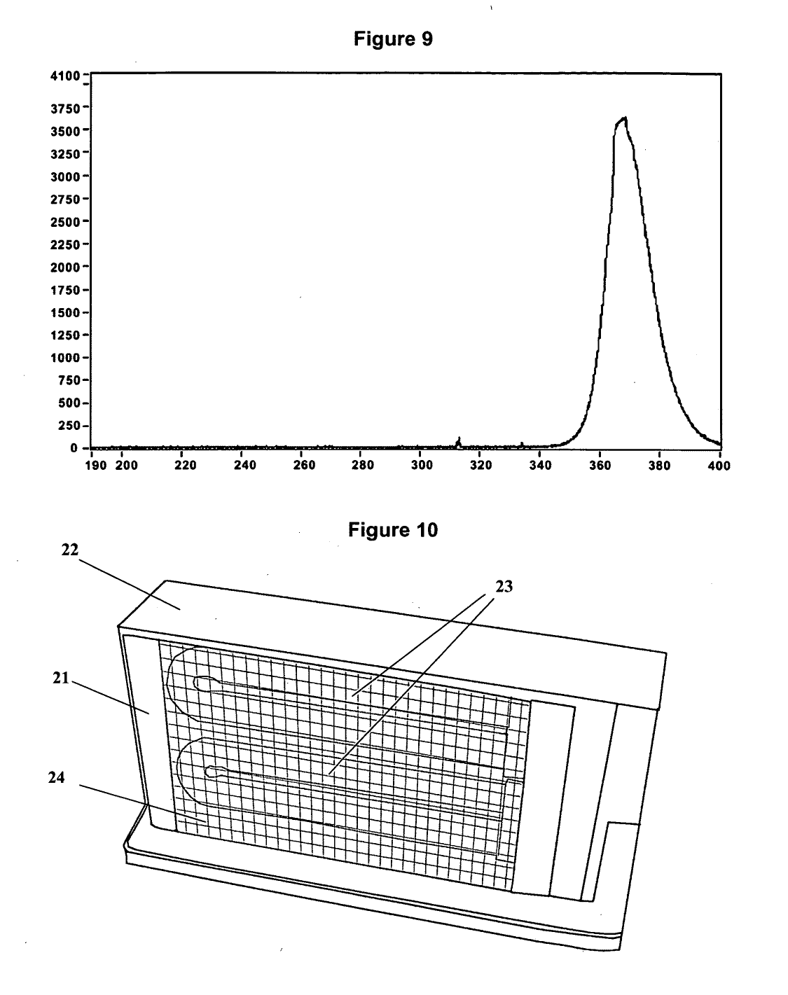

The embodiment of FIG. 7 uses uncoated black-light blue UVA lamps. FIG. 9 shows the spectral emission of the lamps of the embodiment of FIG. 7.

FIG. 10 is the drawing of another embodiment of the invention showing a dismantling cartridge 21 loaded into an air purifier chassis 22 with cover removed, such cartridge containing two photocatalytic semiconductor coated 15 watt black-light white UVA lamps, (FUL15T6), 23 positioned within the cartridge between two outer, photocatalytic semiconductor coated dielectric fiber medium mesh screens located at the cartridge entrance 24 and exit 25. FIG. 11 is the drawing of an image confirming production of free radicals on the rear exit dismantling mesh 25 of the FIG. 9 cartridge, with the cross marks appearing when the clear painted Nitroblue Tetrazoliumon dielectric mesh was activated by hydroxyl radicals produced thereon and displayed the color pattern 26 shown.

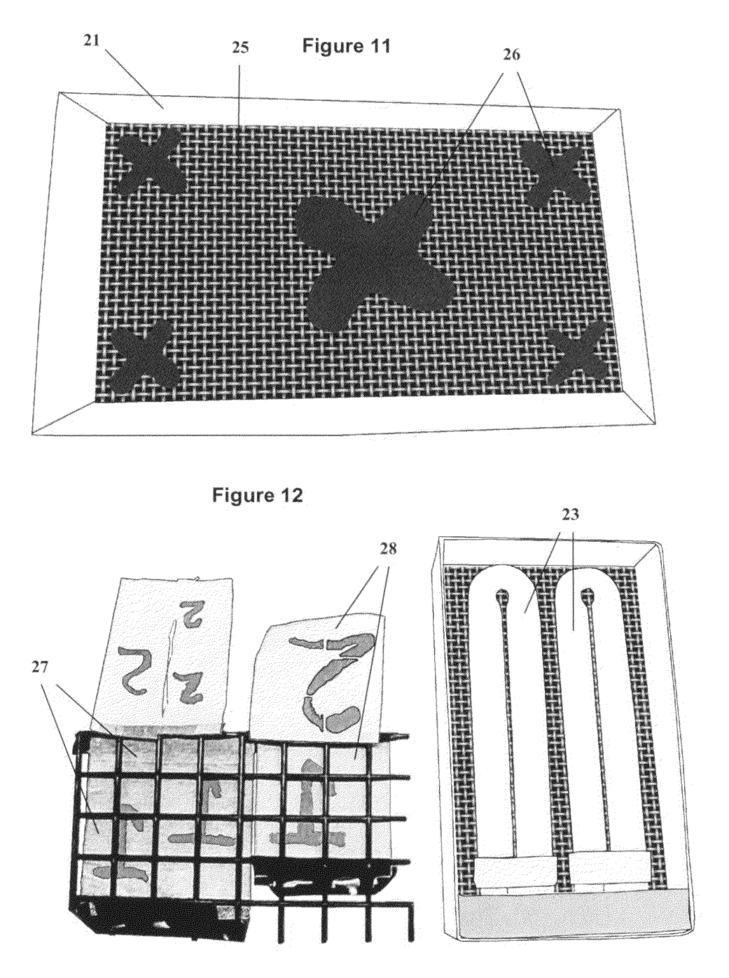

FIG. 12 is a drawing showing free radical creation confirmed by clear Nitroblue Tetrazolium pattern on two each layers of two separate meshes. The mesh pair 27 on the left is constructed of photocatalytic semiconductor coated fluropolymer (Teflon®), PTFE extruded monofilament of 152 microns diameter, 300 microns mesh opening. The mesh pair on the right 28 is constructed of photocatalytic semiconductor coated aluminum, 40 mesh per inch of woven 0.010 inch wire diameter in front of 120 mesh per inch of woven 0.004 inch wire diameter. The mesh layers labeled “1” on 27 and 28 were positioned between the black-light white photocatalytic semiconductor coated UV lamps 23 and mesh layers labeled “2” on 27 and 28, such numbers appearing when the clear painted Nitroblue Tetrazoliumon on the mesh layers were activated by hydroxyl radicals produced thereon and displayed the color patterns shown. These experiments confirm the production of free radicals on photocatalytic semiconductor coated meshes constructed of fluropolymer and aluminum after the UVA energy has passed through a coating on the lamp wall and a coated primary porous dismantling layer to a second activated layer.

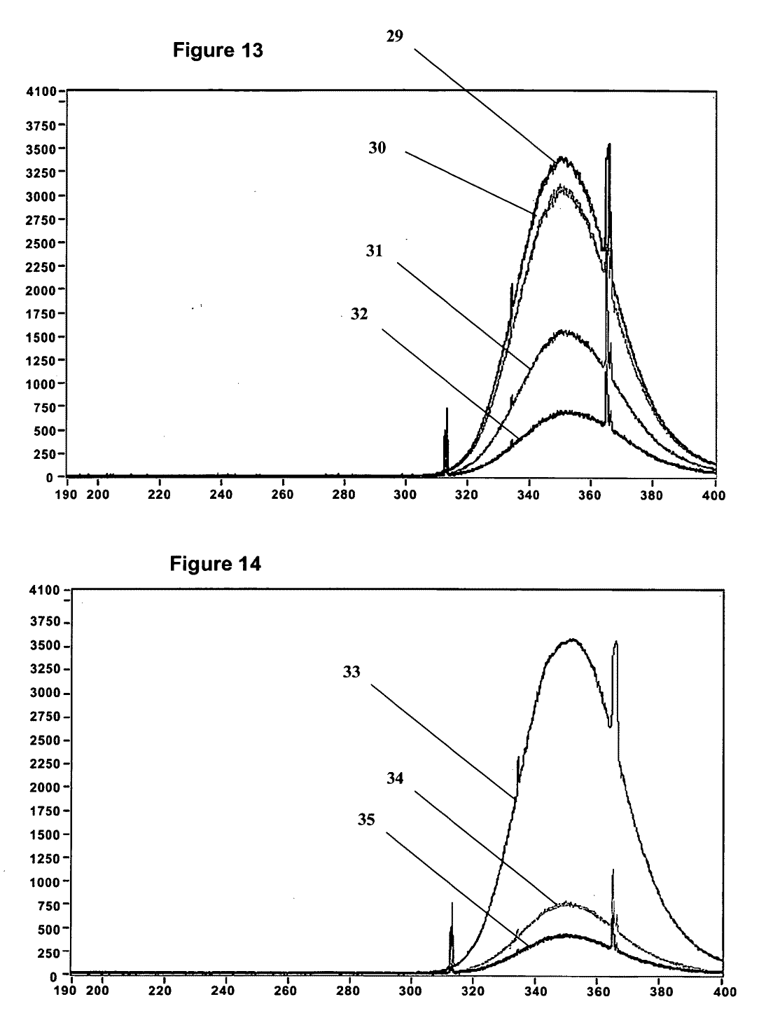

FIG. 13 shows the transmission of UV energy 29 by the coated 50 watt UVA lamp of FIG. 1 through one layer 30 of uncoated fluropolymer mesh 17 of FIG. 8, and separately shows the UV energy transmission through two coated layers 31 and 32 of the fluropolymer mesh 17 of FIG. 8.

Another embodiment of this invention uses a current carrying attraction mesh, such as an electro-conductive polymer or stainless steel, with an outer layer of inorganic dielectric layer applied, that also has an outer layer of photocatalytic semiconductor bonded to the dielectric. FIG. 14 shows the transmission of UV energy 33 by the coated 50 watt UVA lamp of FIG. 1 through two each layers of photocatalytic semiconductor bonded over fluropolymer coated 40 mesh per inch, 0.010 inch wire diameter stainless steel mesh, without any voltage charge applied to the metal core of either layer, where the mesh 34 was positioned adjacent to the uncoated black-light white UVA lamp, and mesh layers 35 was positioned sequentially adjacent thereafter to layer 34. Even through a ultra-fine stainless steel mesh UV energy is still being transmitted to a second layer.

DC voltage in the lower end of the range +140 to +3000 applied at very low current to the current carrying mesh of FIG. 14, attract naturally charged pathogenic contaminants. Voltage on the dismantling mesh can be programmed to reverse automatically on a scheduled cycle to keep the dismantling mesh surface clean, and to enhance the apparatus capacity to eradicate larger particles that may normally carry an inherent positive charge, rather than negative charge.

When the embodiments of this invention are applied to air purification, the suspended contaminants are structurally dissociated and destroyed on the dismantling surfaces, which include the lamp outer wall, the dismantling meshes, and even the dismantling cartridge inner housing. Ambient water vapor is converted to a surface field of hydroxyl radicals by the internal UVA lamp, whose illumination passes through multiple attracting screens. The constantly replenished surface free radicals structurally dissociate the migrated, impacting contaminants into smaller and smaller fragments, eventually to natural stable molecules and harmless protein fragments. Purified air exits the apparatus.

The invention can be in the form of an array of large dimension dismantling mesh cartridges, self-illuminated from within each cartridge, for application to either portable or high volume air flow in fixed HVAC (Heating, Ventilation, Air Conditioning) systems. Cartridges composed of multiple dismantling meshes are suitable for large scale placement in the waste air stream of industrial manufacturing facilities that emit corrosive pollutants or combustible effluents to the atmosphere. Contaminated airflow is purified in each cartridge as photocatalytically active dismantling surfaces eradicate suspended organic and inorganic contaminants impacting them.

What is claimed is:

1. A method for purifying air, said method comprising the steps of
a. directing an air stream carrying contaminants over a lamp outer wall with a dismantling outer surface;
b. further directing said air stream carrying contaminants over a surrounding solid dismantling surface that may optionally be current carrying;
c. further directing said air stream carrying contaminants through a porous dismantling surface that may optionally be current carrying;
d. providing an electrostatic attraction feature to the dismantling surfaces that causes passing contaminants to be migrated to and retained on said dismantling surfaces;
e. directing electromagnetic energy to all dismantling surfaces from within the lamp through the lamp's outer dismantling surface;
f. generating hydroxyl radicals on the dismantling surfaces utilizing said electromagnetic energy; and
g. utilizing said hydroxyl radicals to attract hydrogen molecules from said contaminants.

2. An air purifier comprising:
a. a chamber having an air inlet and air outlet;
b. one or more energy source UVA lamps whose outer wall may have a dismantling surface applied, such lamps disposed in the chamber between the air inlet and air outlet;
c. one or more porous dismantling surfaces disposed in the chamber between the air inlet, the dismantling lamps, and the air outlet, such porous surfaces comprised of either:
i. inorganic dielectric material, or
ii. a current carrying material which may be coated with an inorganic dielectric material.
d. a photocatalytic semiconductor coating applied to the outer surface of said lamps, to porous surfaces within the apparatus, and to the inner apparatus wall.

3. The method of claim 1, wherein the chemical raw materials used to produce the hydroxyl radicals are natural elements contained in the ambient air passing through the apparatus, including water vapor and the gaseous elements in pure air, or supplement feed gas consisting of oxygen, water vapor, and/or hydrogen peroxide.

4. The method of claim 1, wherein the dismantling surfaces have a coating applied of photocatalytic semiconductor nanoparticles embedded in a fixed porous colloid matrix, which coating creates hydroxyl radicals in the porous matrix when exposed to electromagnetic energy and ambient water vapor.

5. The method of claim 1, wherein hydroxyl radicals on and in the activated photocatalytic semiconductor coating structurally dissociate contaminant particles and toxic chemical compounds suspended in the airstream.

6. The method of claim 1, wherein the electromagnetic energy used to activate the photocatalytic semiconductor coating consists of photons in the UVA, UVB, or UVC wavelengths of ultraviolet radiation.

7. The method of claim 1, wherein the ultraviolet electromagnetic energy source is supplied by conventional UV plasma lamps using electrodes embedded in the lamp walls, or from electrode-less UV plasma lamps driven by microwave energy.

8. The method of claim 1, wherein the ultraviolet energy used in the apparatus is low power consumption black-light white, or black-light blue lamps operating in the UVA range.

9. The method of claim 1, wherein the porous dismantling surface is constructed of tetrafluoroethylene or other durable, inorganic dielectric material that is highly transmissive to UV radiation in the 200-400 nm wavelength range necessary for photocatalytic activation.

10. The method of claim 1, wherein the attraction porous dismantling surface is constructed of a current carrying material such as aluminum, copper, brass, tungsten, nickelized steel, or electro-conductive polymer which has in turn been coated with an inorganic, UV transmissive dielectric material such as tetrafluoroethylene.

11. The method of claim 1 wherein a voltage applied to the current carrying attraction surface is sufficient to attract, through its dielectric layer, the natural electrostatic charge on the surface of contaminants entering the apparatus, causing them to migrate to and remain at the photocatalytic semiconductor surface coating on the dielectric layer.

12. The apparatus of claim 2, wherein the UV lamps have a photocatalytic semiconductor coating permanently adhered to the outside wall, such coating being partially transmissive to the UV radiation produced by the lamp plasma, that radiates through the lamp wall and outer coating, and out to other photocatalytic semiconductor coated eradication surfaces.

13. The apparatus of claim 2, wherein the compartment housing the porous dismantling surfaces and the coated lamp, also has a photocatalytic semiconductor coating applied to the compartment walls that are exposed to UV radiation.

14. The apparatus of claim 2, wherein multiple porous dismantling surfaces are arrayed adjacent to one another such that sufficient UV radiation passes through each mesh to illuminate and activate other meshes and enclosure walls upstream or downstream from the lamps.
